(19) United States
(12) Patent Application Publication
(10) Pub. No.: US 2018/0256146 A1
(43) Pub. Date: Sep. 13, 2018

(54) ACCESS DEVICE FOR ANTERIOR CERVICAL DISCECTOMY & FUSION (ACDF)

(71) Applicant: Camino Innovation Holdings LLC, Carlsbad, CA (US)

(72) Inventors: Eugene Chen, Carlsbad, CA (US); Stephanie Frimond, Carlsbad, CA (US); Richard C. Ewers, Carlsbad, CA (US)

(21) Appl. No.: 15/918,744

(22) Filed: Mar. 12, 2018

Related U.S. Application Data
(60) Provisional application No. 62/470,780, filed on Mar. 13, 2017.

(51) Int. Cl. A61B 17/02

(52) U.S. Cl. CPC A61B 17/0218; A61B 2017/00867; A61B 2017/0225

(57) ABSTRACT

A surgical access sleeve may include a tubular braid with a closed end pocket at the distal end. The tubular braid is compressible, or it can be shortened, in a longitudinal direction, to force the tubular braid to expand radially outward. A surgical method includes inserting a substantially un-compressed access sleeve partially into an incision or opening in a patient, with the access sleeve comprising a tubular braid. The access sleeve is compressed in a longitudinal direction to force the access sleeve to expand radially outward to dilate or maintain the opening.

### CROSS-REFERENCE TO RELATED APPLICATION(S)

This application claims priority to U.S. Provisional Patent Application No. 62/470,780 filed Mar. 13, 2017, now pending and incorporated herein by reference.

### TECHNICAL FIELD

The field of the invention is medical systems, devices, and methods for accessing Anterior Cervical Discectomy and Fusion.

### BACKGROUND

Anterior cervical discectomy and fusion (ACDF) is a surgery to remove a herniated or degenerative disc in the neck. An incision is made in the throat area to reach the front of the spine. The disc is removed, and a graft is inserted to fuse together the bones above and below the disc. ACDF surgery may be recommended if physical therapy or medications fail to relieve neck or arm pain caused by pinched spinal nerves. Patients typically go home the same day. Recovery time is generally about four weeks.

Degenerative disc disease causes the discs to dry out. Tears in the disc annulus can allow the gel-filled nucleus material to escape and compress the spinal cord causing numbness and weakness. Bone spurs may develop which can lead to a narrowing of the nerve root canal (foraminal stenosis). The pinched spinal nerve becomes swollen and painful.

Discectomy literally means “cutting out the disc.” A discectomy can be performed anywhere along the spine from the neck (cervical) to the low back (lumbar). The surgeon reaches the damaged disc from the front (anterior) of the spine through the throat area. By moving aside, the neck muscles, trachea, and esophagus, the disc and bony vertebrae are exposed. Surgery from the front of the neck is more accessible than from the back (posterior) because the disc can be reached without disturbing the spinal cord, spinal nerves, and the strong neck muscles. Depending on the symptoms, one disc (single-level) or more (multi-level) may be removed.

After the disc is removed, the space between the bony vertebrae is empty. To prevent the vertebrae from collapsing and rubbing together, a spacer bone graft is inserted to fill the open disc space. The graft serves as a bridge between the two vertebrae to create a spinal fusion. The bone graft and vertebrae are fixed in place with metal plates and screws. Following surgery, the body begins its natural healing process and new bone cells grow around the graft. After 3 to 6 months, the bone graft should join the two vertebrae and form one solid piece of bone.

Bone grafts come from many sources. Each type has advantages and disadvantages. Autograft bone comes from the patient. The surgeon takes bone cells from the patient's hip (iliac crest). This graft has a higher rate of fusion because it has bone-growing cells and proteins. The disadvantage is the morbidity and pain in the hipbone after surgery. Harvesting a bone graft from the hip is done at the same time as the spine surgery. The harvested bone is about a half inch thick—the entire thickness of bone is not removed, just the top half layer. Allograft bone comes from a donor (cadaver). This graft does not have bone-growing cells or proteins, yet it is readily available and eliminates the need to harvest bone from the patient's hip. Allograft is shaped like a doughnut and the center is packed with shavings of living bone tissue taken from the spine during surgery.

Bone graft substitute comes from man-made plastic, ceramic, or bio resorbable compounds. Often called cages, this graft material is packed with shavings of living bone tissue taken from the patient's spine during surgery.

After fusion there may be some range of motion loss, but this varies according to neck mobility before surgery and the number of levels fused. If only one level is fused, there may be similar or even better range of motion than before surgery. If more than two levels are fused, there may be noticeable limits in turning the head and looking up and down. Motion-preserving artificial disc replacements have emerged as an alternative to fusion. Similar to knee replacement, the artificial disc is inserted into the damaged joint space and preserves motion, whereas fusion eliminates motion. Outcomes for artificial disc compared to ACDF are similar, but long-term results of motion preservation and adjacent level disease are not yet proven.

The cervical anterior approach (ACDF Surgery) is started from the anterior part of the neck through a horizontal incision, generally following a wrinkle or a channel on the neck, frequently on the right side once the patient is on the operating table under total anesthesia. The incision generally is about a 3 cm wide except when operating several cervical levels.

The surgeon will push the trachea, esophagus, and neck arteries to the side to be able to access the anterior cervical spine. The neck muscles are drawn apart to clearly uncover the cervical spine so that the surgeon can determine the affected discs, using intraoperative radiology. Typically retractors are used to maintain the surgical access in an open state for a surgeon to operate under direct or microscopic vision. This technique has several disadvantages, including: significant esophageal pain and dysphagia post procedure; difficulty to “steer” the access to a higher or lower disk; and exposure of the anatomy along the surgical channel is large and unprotected.

### SUMMARY

Apparatus and methods for access and exposure in anterior cervical discectomy and fusion (ACDF) surgery are provided.

An access device for ACDF surgery may have the following improvements: low profile insertion; complete 360 degree radial expansion/dilation; protection of the anatomy along the surgical channel; and conformability to the anatomy.

A surgical sleeve may include a tubular braid having a distal end and a proximal end, with a closed end pocket at the distal end. The tubular braid is compressible, or it can be shortened, in a longitudinal direction, to force the tubular braid to expand radially outward.

A surgical method includes inserting a substantially uncompressed access sleeve partially into an incision or opening in a patient, with the access sleeve comprising a tubular braid. The access sleeve is compressed in a longitudinal direction to force the access sleeve to expand radially outward to dilate or maintain the opening.

### BRIEF DESCRIPTION OF THE DRAWINGS

FIG. 1 is a side perspective view of a composite access sleeve made from a self-expanding composite structure possessing a close ended stylet pocket in the distal end.

FIG. 2 is a side perspective view of a stylet inserted into the composite sleeve of FIG. 1.

FIG. 3 is a top perspective view of the composite sleeve tensioned on the stylet of FIG. 2. The sleeve may be tensioned to retract, flatten or be provided with an oval shape on the stylet for insertion.

FIG. 4 is a top perspective view of the composite sleeve partially inserted into an incision.

FIG. 5 is a top perspective view of the composite sleeve of FIGS. 1-4 now fully inserted into the incision.

FIG. 6 is a side perspective view of another embodiment of a radial dilating sleeve having a self-expanding structure made from a braid that is inverted or folded back on itself to provide a double layer of braid material.

FIG. 7 is an enlarged side perspective view of the radial dilating sleeve of FIG. 6 provided with a non-fraying atraumatic distal tip.

FIGS. 8A and 8B are top perspective views of the radial dilating sleeve of FIG. 10 inserted into an incision, but not compressed.

FIGS. 9A and 9B are top perspective views of the radial dilating sleeve of FIGS. 8A and 8B inserted into the incision and now compressed to expand the incision.

FIG. 10 is a side perspective view of another embodiment of a radial dilating sleeve made of an inverted braid tube with opposing tensioning sutures threaded between the braid surfaces.

FIG. 11 is an alternative view showing the radial dilating sleeve of FIG. 10 in a non-tensioned compressible, extendable, minimal radial dilation strength configuration.

FIG. 12 is a side perspective view of the radial dilating sleeve of FIG. 10 shown in a deployment with forceps configuration.

FIG. 13 shows the sleeve of FIGS. 10-12 now retracted and radially expanded with maximum radial dilation strength

FIG. 14 shows the sleeve of FIGS. 10-12 tensioned eccentrically for steering by using one suture providing a positionable distal end.

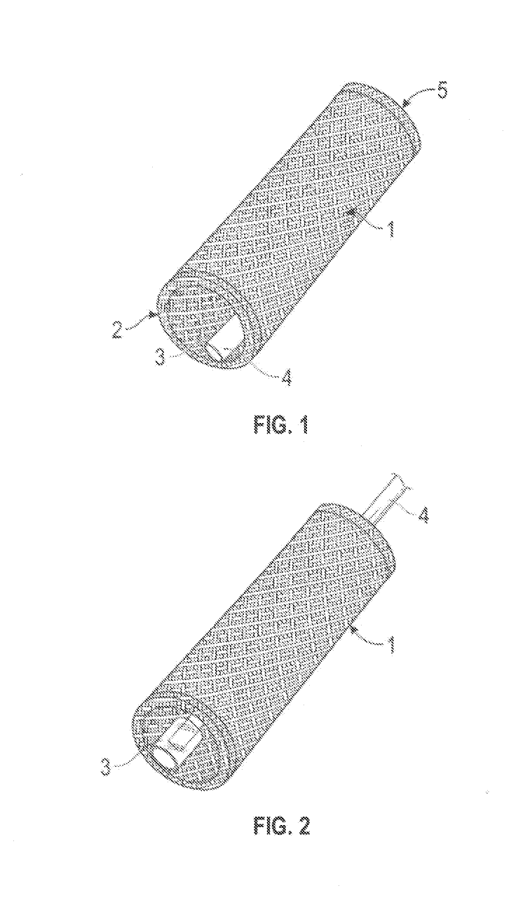

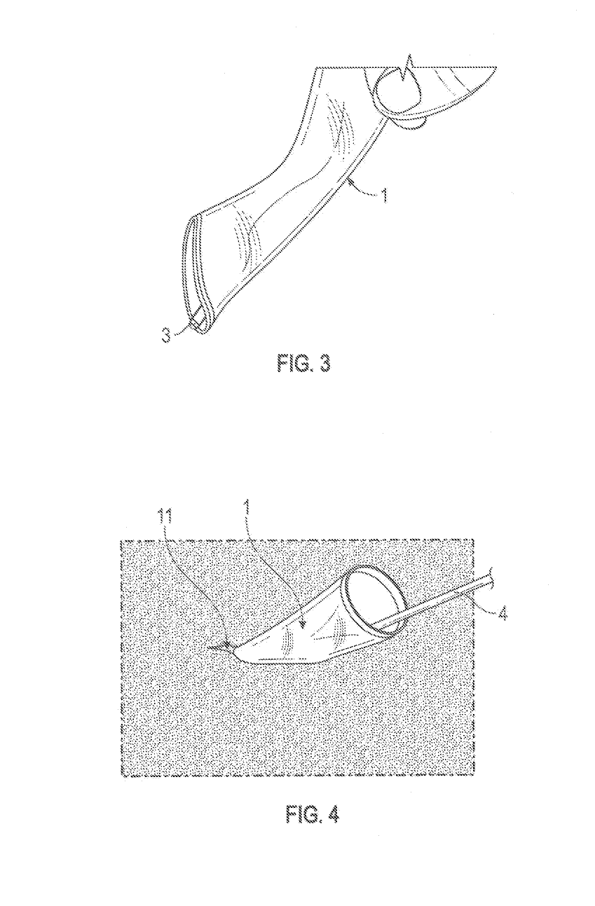

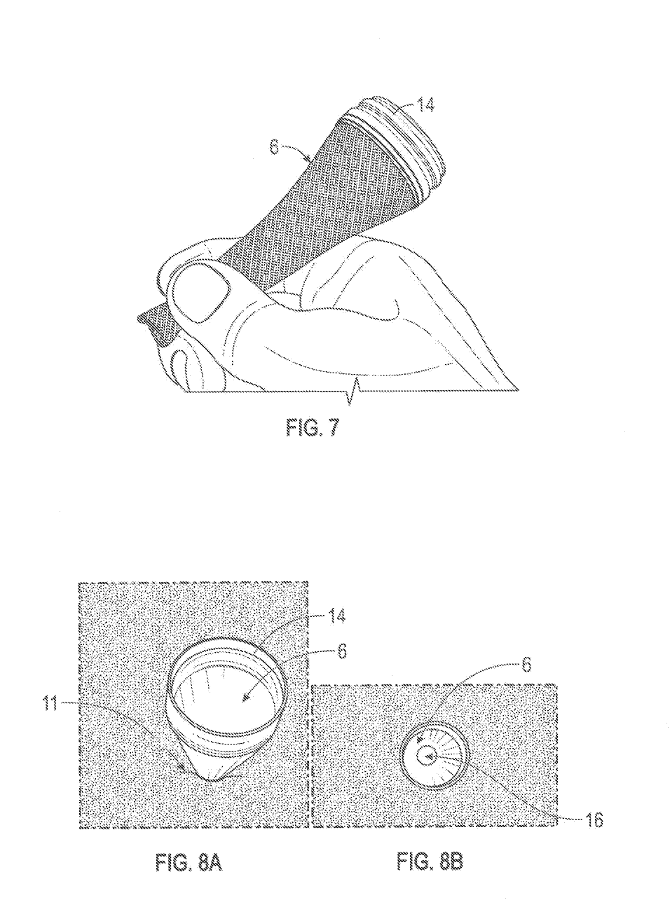

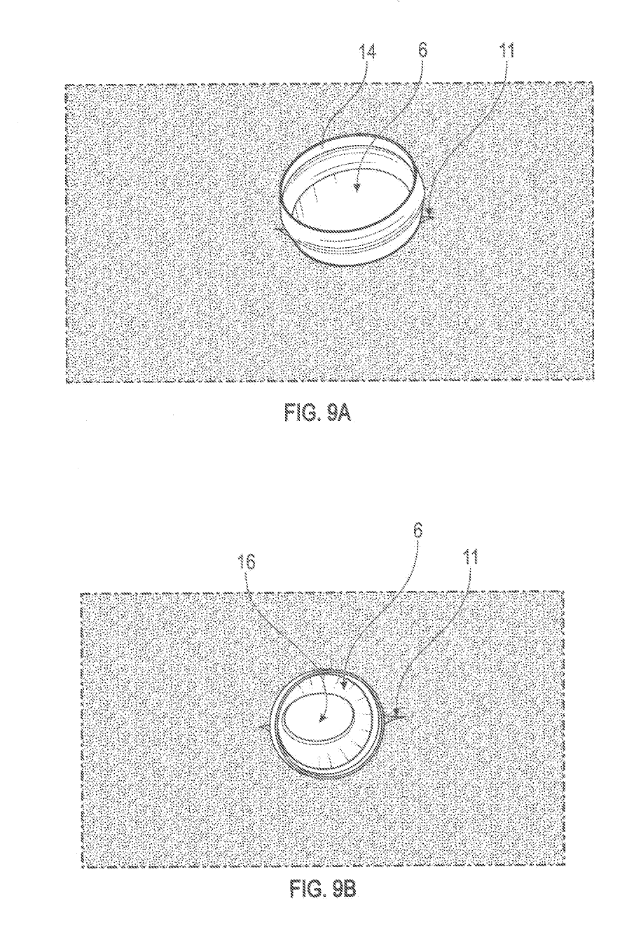

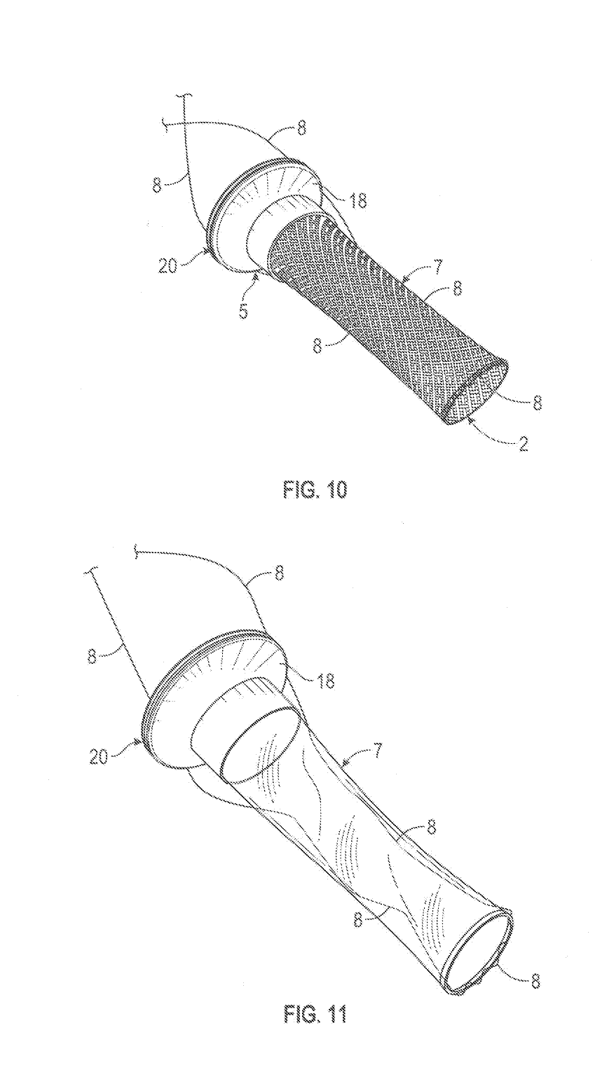

In FIGS. 3-5 for ease and clarity of illustration the sleeve is shown without the crosshatching shown in FIGS. 1 and 2, although the same sleeve is shown in FIGS. 3-5. Similarly, in FIGS. 8A-9B the sleeve is shown without crosshatching, although the same sleeve is shown in FIG. 7. As well, for ease and clarity of illustration the sleeve in FIGS. 11-14 is shown without crosshatching, although the same sleeve is shown in FIG. 10. The arrow L-L in FIG. 13 indicates the longitudinal direction.

### DETAILED DESCRIPTION

The following list of example features corresponds with the figures and is provided for ease of reference, where the same reference number indicates the same element in each of the views.
Composite Dilating Sleeve 1
Distal End of Dilating Sleeve 2
Closed Ended Pocket 3
Stylet 4
Proximal End of Dilating Sleeve 5
Radial Dilating Sleeve Made of Inverted Braid 6
Radial Dilating Sleeve Made with Inverted Braid and Possessing Tensioning Sutures 7
Tensioning Suture 8
Incision or opening 11
End ring 14
Passageway 16
Hub 18
Tensioning Maintaining Slot 20
Insertion Tool 22

A dilating sleeve 1 provides a less traumatic, less cumbersome, less costly, and disposable alternative which also potentially offers more degrees of freedom of instrument motion. The dilating sleeve 1 may be self-expanding. In one embodiment, the sleeve may be fabricated out of a polymer or nitinol braided sleeve. It may be heat set to a desired shape and diameter. Preferably it is coated in an elastomer such as urethane or silicone to make it liquid tight and provide additional structural support, radial dilation strength, and resiliency. Radial dilation strength is the ability of the sleeve to exert outward radial force, to maintain or dilate an opening or incision during surgery.

The sleeve may be positioned in a surgically made incision such that it splints the incision open. This has the advantage of not requiring a mounting structure or control handle that can get in the way of the surgeon's manipulation of instruments. The sleeve may also have a thinner wall than a typical retractor. Additionally, the sleeve may conform to the tissue with 360 degrees of coverage, resulting in less traumatic dilation as the dilating forces are spread across the entire incision. The sleeve may have some ability to give if contacted by portions of the incision anatomy that are particularly resistant to dilation. In this way the sleeve 1 does not force the anatomy to dilate beyond the designed expansion strength of the sleeve. This can result in less incisional trauma.

### Embodiment 1: Semi-Flexible Composite Access Sleeve with Radial Strength

Referring to FIG. 1, a sleeve 1 is configured from a braid with an elastomeric jacket. This construction has the following benefits:
1) Thin wall but possessing radial dilation strength. For example, the sleeve has a radial dilation strength (ability to resist compression forces exerted by the tissue around the incision acting radially inwardly on the sleeve) of 1-20 or 2-10 Newtons.
2) Flexible/foldable/stretchable for a low-profile insertion.

Low friction due to the braid providing a non-continuous surface. The braid surface is essentially a series of discrete contact points thereby reducing the contact area with inserted instrumentation and hence reducing contact friction. Reducing friction may be an important feature for the access sleeve. Inherently materials that can be flexible for changes in shape such as the ability to be elongated into a low profile insertion state and then expand into a high profile working state may be made of a low durometer elastomer. Low durometer elastomers are inherently tacky and result in drag against surgical tools that contact them during surgical manipulation. The braided and or thinly coated braided structures described have shown low friction in engineering testing. A preferred frictional performance of the access sleeve is quantified as having less than or equal to 70 grams of drag force in either push or pull directions against a stainless steel cylindrical instrument of 5 mm diameter when side loaded against the material with a load of 4.5 Newtons. Additionally, it is preferred that this frictional performance is durable to remain relatively unchanged through a minimum of 25 interactions of the surgical tool. Additionally, it is preferred and achieved with this structure that no additional lubricious coatings or application of lubricant is required. Surface coatings are less preferred because they may wear away during use, require activation for example by water, and add cost. Lubricants are less preferred because they add potential mess to the surgical access sight, may migrate to target tissue and make manipulation of the tissue more difficult, and may migrate to the instrument handle or surgeon's hands causing potential difficulties in control and manipulation of the instruments.

Referring to FIG. 2, a stylet 4 is inserted into a closed end pocket 3 at the distal end of the composite access sleeve 1.

Referring to FIG. 3, the composite access sleeve 1 is stretched on the stylet 4 causing the sleeve 1 to neck down and form a low profile oval shape.

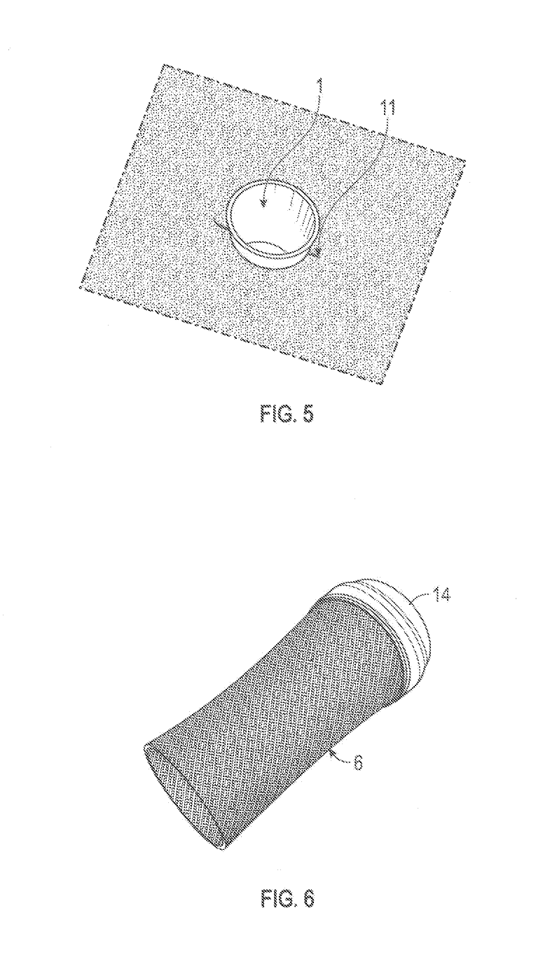

Referring to FIG. 4, composite access sleeve 1 is introduced into a surgical incision using the stylet 4 to push the distal end of the sleeve into the incision 11. FIG. 5 shows the composite access sleeve 1 in position in the incision.

### Embodiment 2—Radial Dilating Sleeve

Referring to FIG. 6, an inverted braid provides a radially dilating sleeve 6. The sleeve 6 has the advantage of being able to be elongated into a necked down configuration and then expanded by compression. The sleeve 6 is made of a braid that has been folded/inverted to create a non-fraying leading edge or end 2. An end ring 14 may be provided on the proximal end of the sleeve 6.

Referring to FIG. 7, the sleeve 6 is shown partially elongated to a low profile for insertion into a surgical incision.

Referring to FIG. 8A and FIG. 8B, the sleeve 6 is introduced into a surgical incision but still in its elongate configuration. A portion of the length of the sleeve 6, typically 50 to 90% of the entire length of the sleeve, may extend out of the surgical incision (or a length of about 60-110 or 70 to 100 mm). In this embodiment, the sleeve 6 may have a small passageway 16 with a diameter of about 2-20 or 4-10 mm.

Referring to FIGS. 9A and 9B, the sleeve 6 is pressed into the surgical incision. In contrast to FIGS. 8A and 8B, here only a small fraction of the sleeve 6 extends out of the incision 11, typically 5-30% of the entire length of the sleeve 6, or a length of about 5-30 mm. Correspondingly, the sleeve 6 has a larger passageway 16 with a diameter of 20-40 mm, because the braid has radially dilated the surgical incision.

### Embodiment 3—Radial Dilating Sleeve with Tensioning Sutures

Referring to FIG. 10, a radial dilating sleeve may be made of an inverted braid 7 (i.e., a tube of braid material turned back on itself to provide a double wall thickness of braid material). A suture 8 is threaded between the inner and outer braid surfaces. The suture 8 is attached to the braid 7 at the distal tip 2 of the braid. The suture 8 runs outside of the braid walls at the proximal end 5. A hub 18 with a slot 20 is provided at the proximal end 5. The slot 20 receives and frictionally holds the tensioning sutures 8.

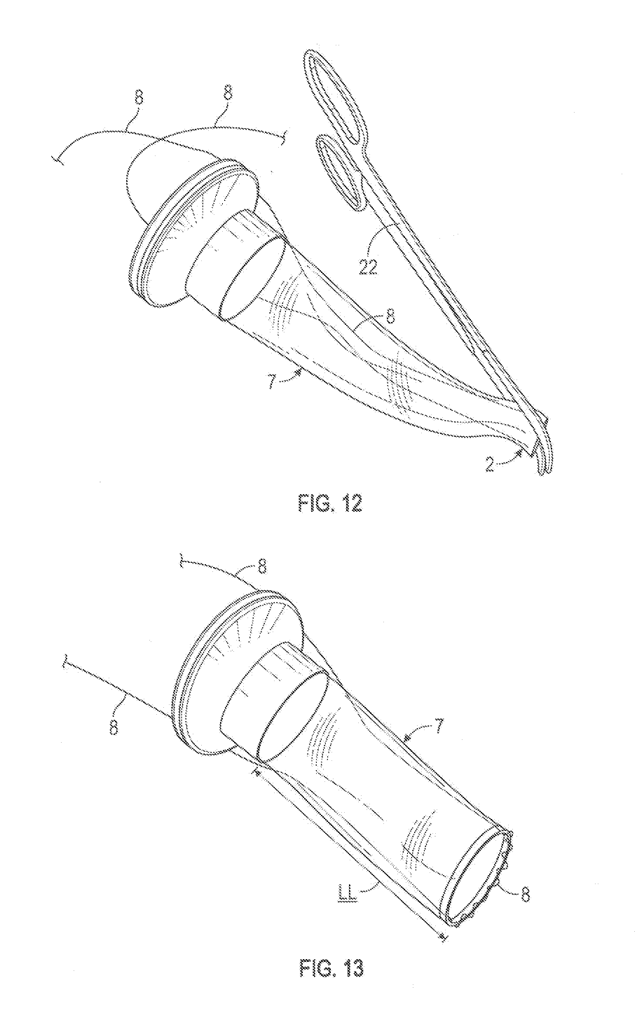

In each of the embodiments described the user can actively control the radial dilation strength of the sleeve by configuring the sleeve with tensioning sutures. This allows the user to have the device in an initial un-tensioned configuration as shown in FIG. 10. In the un-tensioned configuration the device is in its most flexible and elongate state. In this state the device may be elongated and compressed by hand for insertion into an incision. Preferably it may be compressed and elongated by an insertion tool or forceps 22 for placement as shown in FIG. 12. The sleeve may be compressed from its initial un-tensioned length LL, to a tensioned configuration having a length of 20, 30, 40, 50, or 60% of LL.

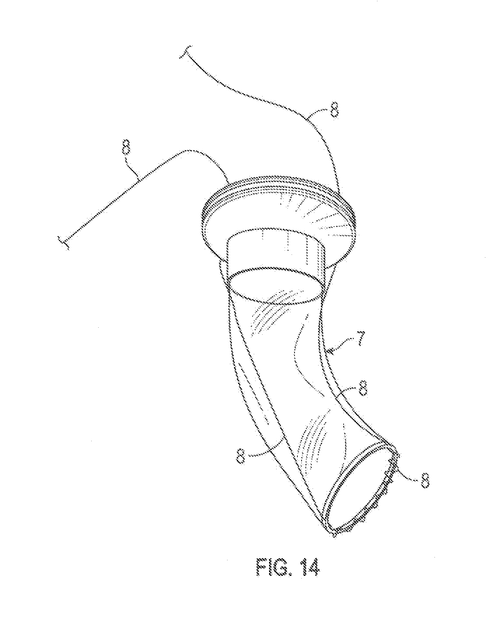

Once inserted, the user can apply tension to sutures 8 to impart a relative compression between the distal and proximal ends 2 and 5. This compression causes the braid sleeve to be configured into its maximum radial dilation strength state as shown in FIG. 13. Additionally, the tension can be applied eccentrically, one suture tensioned more than the other, to impart an articulation to the distal tip as shown in FIG. 14.

The sleeve in the form of a tubular braid may be actively tensioned using two or more evenly spaced ratcheting strips (zip ties) attached at the distal rim and extending from the proximal collar.

Thus, novel apparatus and method have been shown and described. Various changes and substitutions may of course be made without departing from the spirit and scope of the invention. The invention, therefore, should not be limited, except by the following claims, and their equivalents.

We claim:

1. A surgical method, comprising:
inserting a substantially un-compressed access sleeve partially into an opening in a patient, with the access sleeve comprising a tubular braid; and
compressing the access sleeve in a longitudinal direction to force the access sleeve to expand radially outward to dilate or maintain the opening.

2. The method of claim 1 wherein the opening is a surgical incision.

3. The method of claim 2 further including inserting a surgical instrument through the compressed access sleeve.

4. The method of claim 1 wherein the opening is a natural opening in the body of the patient.

5. The method of claim 1 wherein the tubular braid comprises polymer or nitinol.

6. The method of claim 1 wherein the access sleeve has a radial dilation strength of 2-10 Newtons.

7. The method of claim 1 wherein the tubular braid is folded over on itself to form a sleeve having a double wall structure.

8. The method of claim 7 with the double wall structure having an atraumatic and fray resistant tip due to the folding over of the braid material.

9. The method of claim 1 further comprising compressing the sleeve by tensioning at least one suture attached to a distal end of the sleeve.

10. The method of claim 9 further comprising tensioning at least two sutures attached to the distal end the sleeve.

11. The method of claim 10 further comprising steering a distal end of the sleeve via the tensioning of the at least two sutures attached to the distal end the sleeve.

12. The method of claim 2 wherein the surgical incision is in the throat of the patient for performing an anterior cervical discectomy and fusion.

13. The method of claim 1 further including inserting the access sleeve into the opening by inserting a surgical tool into a pocket at a distal end of the access sleeve, and pushing the sleeve into the incision using the surgical tool.

14. A surgical method, comprising:
inserting a substantially un-compressed access sleeve partially into an opening in a patient, with the access sleeve comprising a tubular braid; and
allowing the sleeve to contract along its longitudinal axis, with the sleeve simultaneously expanding radially outward to dilate or maintain the opening.

15. The method of claim 14 further comprising stretching the sleeve from an inherently compressed state into the substantially un-compressed state.

16. A surgical sleeve, comprising:
a tubular braid having a distal end and a proximal end;
a closed end pocket at the distal end;
the tubular braid compressible in a longitudinal direction to force the tubular braid to expand radially outward.

17. The surgical sleeve of claim 16 with the tubular braid inverted to provide a double wall thickness.

18. The surgical sleeve of claim 17 further comprising at least one tensioning suture attached to the distal end.

19. The surgical sleeve of claim 17 comprising a first suture on a first side of the tubular braid and a second suture on a second side of the tubular braid, opposite from the first side.

20. The surgical sleeve of claim 18 further including a hub attached to the proximal end, with the hub including a slot for holding the at least one tensioning suture.
